(19) United States
(12) Patent Application Publication
(10) Pub. No.: US 2005/0165346 A1
(43) Pub. Date: Jul. 28, 2005

(54) Double-sided surgical irrigator

(76) Inventors: Geoffrey James Neilson, New South Wales (AU); Anthony Maloof, New South Wales (AU); Edward John Milverton, New South Wales (AU)

Correspondence Address: GOTTLIEB RACKMAN & REISMAN PC, 270 MADISON AVENUE, 8TH FLOOR, NEW YORK, NY, 100160601, US

(21) Appl. No.: 10/524,723

(22) PCT Filed: Aug. 22, 2003
(86) PCT No.: PCT/AU03/01084

(30) Foreign Application Priority Data
Aug. 22, 2002 (AU) 2002951307

(51) Int. Cl. A61B 17/20; A61B 17/32

(52) U.S. Cl. 604/27

(57) ABSTRACT

A surgical irrigation device (10) consists of a continuous outer wall (12) and a continuous inner wall (11) which are held apart and inter-connected by one or more spacers (20). The upper edges (13), (15) of the inner and outer walls (11), (12) define an upper vacuum channel (17) and the lower extremities (14), (16) of the inner wall (11) and outer wall (12) a lower vacuum channel. A vacuum tube (22) leads from outside the body (10) into the space between the inner and outer walls (11), (12). An irrigation tube (23) passes from outside of the body (10), through both the inner walls (11), (12) so as to terminate within the central space defined by the interior wall (11) and an aspiration tube (25) which originates externally to the body passes through both the inner and outer walls (11), (12) at a different location to the irrigation tube (23).

### FIELD OF THE INVENTION

The present invention pertains to surgical irrigation devices and more particularly to an irrigation device which is adapted to isolate a volume in the human body which volume lies between two tissues.

### BACKGROUND OF THE INVENTION

Some surgical and therapeutic treatments of the human eye require particular areas of ocular tissues to be treated with generally toxic fluids. It is therefore highly desirable to limit the areas which are contacted by these fluids. In particular it is desirable to isolate adjacent surfaces or areas which lie on tenon's capsule and the sclera.

### SUMMARY OF THE INVENTION

Accordingly, there is provided an irrigation device comprising a body having a continuous outer wall and a continuous inner wall which are held apart and inter-connected by one or more perforated spacers. The upper edges of the inner and outer walls may each comprise an upper seal. Together, these upper seals may co-operate to define an upper vacuum channel.

The lower extremities of the inner wall and outer wall may also terminate in sealing surfaces which together form a lower vacuum channel. A vacuum tube may lead from outside the body into a space between the inner and outer walls. Vacuum drawn through this tube is distributed evenly about the upper and lower vacuum channels.

An irrigation tube may pass from outside of the body, through both the exterior and interior walls so as to terminate within the central space defined by the interior wall. An aspiration tube which originates externally to the body may also pass through both the interior and exterior wall at a different location to the irrigation tube.

In a preferred embodiment of the invention, the irrigation tube extends into the central space defined by the interior wall so that it is distant from an aspiration port formed in the interior wall.

### BRIEF DESCRIPTION OF THE DRAWINGS

In order that the invention may be more readily understood and put into practical effect, reference will now be made to the accompanying drawings in which:

FIG. 1 is a schematic cross-sectional view of an irrigation device according to one embodiment of the invention located between tenon's capsule and the sclera;

FIG. 2 is a top plan view of the device depicted in FIG. 1;

FIG. 3 is a top plan view of a double-sided irrigation device according to another embodiment of the invention;

FIG. 4 is a bottom plan view of the device depicted in FIG. 3 illustrating the location of irrigation, aspiration and vacuum tubes;

FIG. 5 is a cross-section through lines 5-5 of FIG. 3;

FIG. 6 is a cross-section through lines 6-6 of FIG. 4; and

FIG. 7 is a view similar to FIG. 5 of a further embodiment of the invention illustrating separate vacuum control of the upper and lower vacuum channels.

### PREFERRED EMBODIMENTS OF THE INVENTION

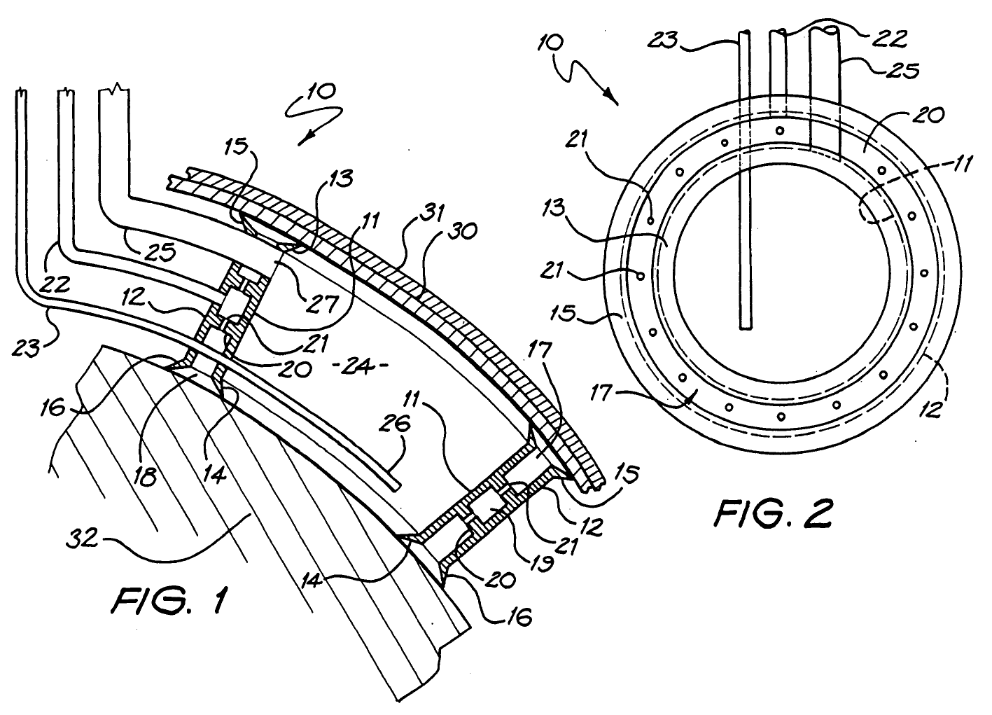

As shown in FIG. 1, a double-sided irrigator 10 according to one embodiment of the invention comprises a continuous inner wall 11 and a continuous outer wall 12. In this instance, the inner and outer walls 11, 12 are approximately the same height and spaced apart from one another equally about their entire periphery. The continuous inner wall 11 terminates at its upper end with an upper seal 13 and at its lower end with a lower seal 14. Similarly, the outer wall 12 terminates at its upper end with an upper seal 15 and at its lower end with a lower seal 16. Together, the upper seals 13, 15 define an upper vacuum channel 17. Similarly, the lower seals 14, 16 define a lower vacuum channel 18.

In this embodiment, each of the seals 13, 14, 15, 16 comprises an enlarged portion, lip or flange over which the pressure associated with sealing is distributed. This minimises the contact pressure and increases the sealing area and effectiveness in those areas where the device contacts tissue.

The space 19 between the inner and outer walls 11, 12 defines a manifold which is adapted to supply equal vacuum pressure to the upper and lower vacuum channels 17, 18. In order that the device be mechanically stable, one or more spacers 20 are interposed and preferably affixed to both the interior surfaces of the inner and outer walls 11, 12. The spacers 20 may be in the form of continuous ring-like structures having perforations 21 which allow the internal pressure to be equalised on either side of the one or more spacers.

Simultaneous sealing between two generally parallel tissues is accomplished by delivering vacuum pressure to the interior 19. This is done from a vacuum tube 22 which originates outside of the outer wall 12 and terminates and communicates fluidically with the interior 19 between the walls. Once the device is sealed in place, a fluid may be introduced through an irrigation tube 23 which originates outside of the exterior wall 12 and passes through both the exterior and interior walls 11, 12 before terminating within the central space 24 surrounded by the interior wall 11.

An aspiration tube 25 is also provided. The aspiration tube 25 passes through the exterior and interior walls 11, 12 and terminates in the central space or cavity 24. In a preferred embodiment, the irrigation tube extends at least partially across the central space or cavity 24 so that the distance between the end 26 of the irrigation tube and the end 27 of the aspiration tube are well separated from one another. This provides the longest practical minimum flow path between the two ends 26, 27.

It will be appreciated that the device as depicted in FIGS. 1 and 2 is capable of a wide variety of uses. In the particular embodiment depicted, the device is conveniently positioned between tenon's capsule 30 (the lining of the conjunctiva 31) and the surface of the sclera 32. It will be appreciated that a device of this kind has a wide variety of uses where it is desirable to isolate a volume between two surfaces or two tissues, particularly where that volume is subject to irrigation and aspiration and more particularly where it is important to maintain that volume as a constant without increasing the pressure within it. It will be appreciated that for surgical or therapeutic procedures of this kind the general thickness of the body will be no more than about 2 mm and that the maximum dimension and plan view (corresponding to the diameter of a circle) will be about 15 mm.

As shown in FIG. 3, a double-sided irrigation device may define different sealing profiles with its upper and lower vacuum channel 41 in this example is essentially circular. The major portion of the upper vacuum channel 42 is generally circular and a minor portion 43 is rebated. Note that the inner 44 and outer 45 upper seals maintain a uniform spacing just as the inner 46 and outer 47 lower seals are uniformly spaced throughout.

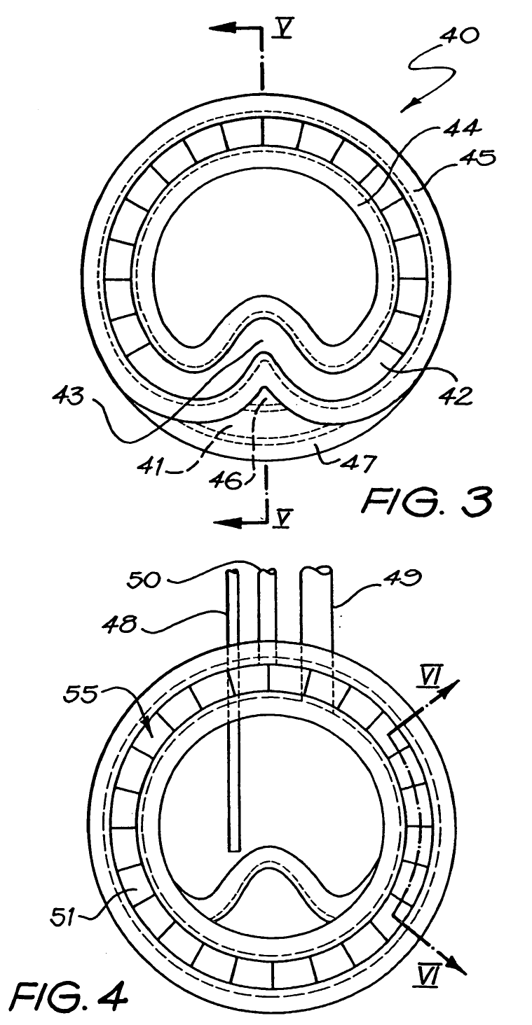

As shown in FIG. 4, a device of the type depicted in FIG. 3 may be provided with an irrigation tube 48 which terminates within the central cavity. An aspiration tube 49 is also provided. In this example, a single vacuum tube 50 terminates between the inner and outer walls and thus introduces vacuum into a central manifold 55 which is located between the inner and outer walls.

As shown in FIG. 5, the upper vacuum channel 42 and the lower vacuum channel 41 are seen to have different configurations. In this example, spacers 51 are interposed between the inner and outer walls where the upper and lower channels coincide. Spacers 51 may be provided with through holes 52 to allow vacuum pressure to be communicated evenly between the upper and lower vacuum channels 41, 42.

As shown in FIG. 6, the spacers 51 may be provided as arrays of discrete blocks or other shapes with no overlap between the spacers 51 of the upper tier 53 and those of the lower tier 54. This is one way of providing sufficient rigidity in the vacuum channel area while still allowing vacuum pressure to be communicated evenly between the upper and lower vacuum channels.

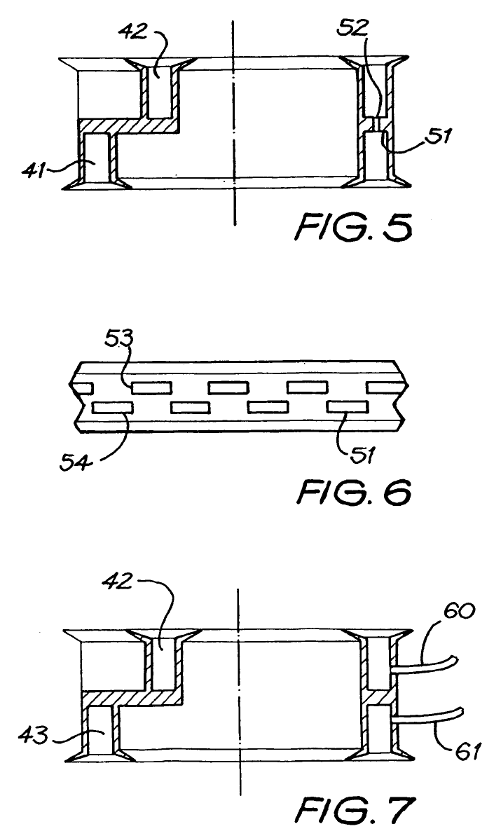

As shown in FIG. 7, the upper vacuum channel 42 and lower vacuum channel 43 may be fluidicly disconnected and supplied with separate vacuum sources 60, 61. In this way, the vacuum to the upper and lower channels 42, 43 may be controlled independently.

The device may be used, for example, in trabeculectomy procedures (for glaucoma) to prevent adhesion of the tissues to the filtering bleb. In preferred embodiments, the device is provided as a disposable instrument, being flexible and injection moulded from an elastomer. However, rigid moulded or machined devices are also contemplated.

It will be appreciated that in addition to sealing a defined profile a flexible device with the same top and bottom sealing profiles may be formed into a shape between the tissues, prior the applying the vacuum thus sealing a shape defined by the user at the time that the vacuum is applied.

It is also envisaged that the aspiration tube or tubes may be optionally connected to a sealed drainage bag for simple disposal of the irrigation liquid which provides for safer handling of toxic chemicals such as cytotoxic drugs.

Although the invention has been disclosed with reference to particular details of construction it will be appreciated that these have been provided by way of example and not as limitations to the scope or spirit of the invention. Specifically, the device has been disclosed, (for example in FIG. 2) as being generally circular. It will be appreciated that the device may be provided in any variety of external confirmations including, without limitation, circular, oval, crescent or irregular confirmations. Various other modifications may be made to the irrigation device without departing from the scope and ambit of the invention.

1. A surgical irrigation device comprising a body having a continuous outer wall and a continuous inner wall which are held apart by one or more spacers, the upper extremities of the inner and outer walls each having an upper sealing surface which together form an upper vacuum channel and the lower extremities of the inner and outer walls each having a lower sealing surface which together form a lower vacuum channel.

2. A surgical irrigation device according to claim 1 and further including a vacuum tube leading from outside the body into the space between the inner and outer walls, and where a vacuum drawn through the vacuum tube is distributed evenly about the upper and lower vacuum channels.

3. A surgical irrigation device according to claim 1 and further including an irrigation tube leading from outside of the body, through both the exterior and interior walls so as to terminate within the central space defined by the interior wall.

4. A surgical irrigation device according to claim 3 and further including an aspiration tube leading from outside of the body through both the interior and exterior walls and terminates at a different location to the irrigation tube.

5. A surgical irrigation device according to claim 3 wherein the irrigation tube extends into the central space defined by the interior wall so that it is distant from an aspiration port formed in the interior wall.

6. A surgical irrigation device according to claim 1 wherein the spacers are perforated.

7. A surgical irrigation device according to claim 1 wherein the spacers comprise first and second tiers of spaced apart spacers with no overlap between the spacers of the first tier and the spacers of the second tier.

8. A surgical irrigation device according to claim 1 wherein there is a boundary between the upper vacuum channel and the lower vacuum channel and further including a first vacuum tube leading from outside the body into the upper vacuum channel and a second vacuum tube leading from outside the body into the lower vacuum channel.

9. A surgical irrigation device according to claim 1 wherein the lower vacuum channel and the upper vacuum channel are of the same configuration.

10. A surgical irrigation device according to claim 9 wherein the lower vacuum channel and the upper vacuum channel are circular in plan.

11. A surgical irrigation device according to claim 1 wherein the lower vacuum channel is circular in plan and the major portion of the upper vacuum channel is circular in plan and the minor portion is rebated.

12. A surgical irrigation device according to claim 1 wherein the upper extremities of the inner and outer walls and the lower extremities of the inner and outer walls comprised enlarged lips.
